SEQUENCE LISTING (72 sequences)

SEQ ID NO: 1 - protein, 14 aa, synthetic construct
  REGION 1..14: note = synthetic peptide
  MOD_RES 1: note = 4-fluorobenzoyl-arginine
  MOD_RES 3: note = 3-(2-naphthyl) alanine
  MOD_RES 6: note = citrulline
  MOD_RES 8: note = D-lysine
  MOD_RES 12: note = citrulline
  MOD_RES 14: note = C' AMIDATED
  DISULFID 4..13: note = may be linked by disulfide bond between Cys
RRACYRKKPYRRCR

SEQ ID NO: 2 - protein, 14 aa, synthetic construct
  REGION 1..14: note = SYNTHETIC PEPTIDE
  MOD_RES 1: note = N' ACETYLATION
  MOD_RES 3: note = 3-(2-naphthyl) alanine
  MOD_RES 6: note = citrulline
  MOD_RES 8: note = D-lysine
  MOD_RES 12: note = citrulline
  DISULFID 4..13: note = may be linked by disulfide bond between Cys
RRACYRKKPYRRCR

SEQ ID NO: 3 - protein, 14 aa, synthetic construct
  REGION 1..14: note = SYNTHETIC PEPTIDE
  MOD_RES 1: note = N' ACETYLATION
  MOD_RES 3: note = 3-(2-naphthyl) alanine
  MOD_RES 8: note = D-citrulline
  MOD_RES 12: note = citrulline
  DISULFID 4..13: note = may be linked by disulfide bond between Cys
RRACYRKRPYRRCR

SEQ ID NO: 4 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 6: note = citrulline
  MOD_RES 8: note = D-citrulline
  MOD_RES 12: note = citrulline
  DISULFID 4..13: note = may be linked by disulfide bond between Cys
RRACYRKRPYRRCR

SEQ ID NO: 5 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 6: note = citrulline
  MOD_RES 8: note = D-lysine
  MOD_RES 11..12: note = citrulline
  DISULFID 4..13: note = may be linked by disulfide bond between Cys
RRACYRKKPYRRCR

SEQ ID NO: 6 - protein, 14 aa, synthetic construct
  REGION 1..14: note = synthetic peptide
  MOD_RES 1: note = N' acetylated citrulline
  MOD_RES 3: note = 3-(2-naphthyl) alanine
  MOD_RES 6: note = citrulline
  MOD_RES 8: note = D-lysine
  MOD_RES 12: note = citrulline
  DISULFID 4..13: note = may be linked by disulfide bond between Cys
RRACYRKKPYRRCR

SEQ ID NO: 7 - protein, 14 aa, synthetic construct
  REGION 1..14: note = synthetic peptide
  MOD_RES 1: note = N' acetylated citrulline
  MOD_RES 3: note = 3-(2-naphthyl) alanine
  MOD_RES 8: note = D-citrulline
  MOD_RES 12: note = citrulline
  DISULFID 4..13: note = may be linked by disulfide bond between Cys
RRACYRKRPYRRCR

SEQ ID NO: 8 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 8: note = D-citrulline
  MOD_RES 11..12: note = citrulline
  DISULFID 4..13: note = may be linked by disulfide bond between Cys
RRACYRKRPYRRCR

SEQ ID NO: 9 - protein, 14 aa, synthetic construct
  REGION 1..14: note = synthetic peptide
  MOD_RES 1: note = N' ACETYLATED citrulline
  MOD_RES 3: note = 3-(2-naphthyl) alanine
  MOD_RES 8: note = D-lysine
  MOD_RES 11..12: note = citrulline
  DISULFID 4..13: note = may be linked by disulfide bond between Cys
RRACYRKKPYRRCR

SEQ ID NO: 10 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 6: note = citrulline
  MOD_RES 8: note = D-citrulline
  MOD_RES 12: note = citrulline
  MOD_RES 14: note = C' AMIDATED
  DISULFID 4..13: note = may be linked by disulfide bond between Cys
RRACYRKRPYRRCR

SEQ ID NO: 11 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 6: note = citrulline
  MOD_RES 8: note = D-lysine
  MOD_RES 11..12: note = citrulline
  MOD_RES 14: note = C' AMIDATED
  DISULFID 4..13: note = may be linked by disulfide bond between Cys
RRACYRKKPYRRCR

SEQ ID NO: 12 - protein, 14 aa, synthetic construct
  REGION 1..14: note = synthetic peptide
  MOD_RES 1: note = N' ACETYLATED citrulline
  MOD_RES 3: note = 3-(2-naphthyl) alanine
  MOD_RES 6: note = citrulline
  MOD_RES 8: note = D-lysine
  MOD_RES 12: note = citrulline
  MOD_RES 14: note = AMIDATED
  DISULFID 4..13: note = may be linked by disulfide bond between Cys
RRACYRKKPYRRCR

SEQ ID NO: 13 - protein, 14 aa, synthetic construct
  REGION 1..14: note = synthetic peptide
  MOD_RES 1: note = N' ACETYLATED citrulline
  MOD_RES 3: note = 3-(2-naphthyl) alanine
  MOD_RES 8: note = D-citrulline
  MOD_RES 12: note = citrulline
  MOD_RES 14: note = C' AMIDATED
  DISULFID 4..13: note = may be linked by disulfide bond between Cys
RRACYRKRPYRRCR

SEQ ID NO: 14 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 8: note = D-citrulline
  MOD_RES 11..12: note = citrulline
  MOD_RES 14: note = C' AMIDATED
  DISULFID 4..13: note = may be linked by disulfide bond between Cys
RRACYRKRPYRRCR

SEQ ID NO: 15 - protein, 14 aa, synthetic construct
  REGION 1..14: note = synthetic peptide
  MOD_RES 1: note = N' ACETYLATED citrulline
  MOD_RES 3: note = 3-(2-naphthyl) alanine
  MOD_RES 8: note = D-lysine
  MOD_RES 11..12: note = citrulline
  MOD_RES 14: note = C' AMIDATED
  DISULFID 4..13: note = may be linked by disulfide bond between Cys
RRACYRKKPYRRCR

SEQ ID NO: 16 - protein, 14 aa, synthetic construct
  REGION 1..14: note = synthetic peptide
  MOD_RES 1: note = D-glutamic acid
  MOD_RES 3: note = 3-(2-naphthyl) alanine
  MOD_RES 8: note = D-lysine
  MOD_RES 12: note = citrulline
  DISULFID 4..13: note = may be linked by disulfide bond between Cys
ERACYRKKPYRRCR

SEQ ID NO: 17 - protein, 14 aa, synthetic construct
  REGION 1..14: note = synthetic peptide
  MOD_RES 3: note = 3-(2-naphthyl) alanine
  MOD_RES 8: note = D-lysine
  MOD_RES 12: note = citrulline
  DISULFID 4..13: note = may be linked by disulfide bond between Cys
REACYRKKPYRRCR

SEQ ID NO: 18 - protein, 14 aa, synthetic construct
  REGION 1..14: note = synthetic peptide
  MOD_RES 3: note = 3-(2-naphthyl) alanine
  MOD_RES 8: note = D-lysine
  MOD_RES 12: note = citrulline
  DISULFID 4..13: note = may be linked by disulfide bond between Cys
RRACYEKKPYRRCR

SEQ ID NO: 19 - protein, 14 aa, synthetic construct
  REGION 1..14: note = synthetic peptide
  MOD_RES 3: note = 3-(2-naphthyl) alanine
  MOD_RES 8: note = D-lysine
  MOD_RES 12: note = citrulline
  DISULFID 4..13: note = may be linked by disulfide bond between Cys
RRACYREKPYRRCR

SEQ ID NO: 20 - protein, 14 aa, synthetic construct
  REGION 1..14: note = synthetic peptide
  MOD_RES 3: note = 3-(2-naphthyl) alanine
  MOD_RES 8: note = D-glutamic acid
  MOD_RES 12: note = citrulline
  DISULFID 4..13: note = may be linked by disulfide bond between Cys
RRACYRKEPYRRCR

SEQ ID NO: 21 - protein, 14 aa, synthetic construct
  REGION 1..14: note = synthetic peptide
  MOD_RES 3: note = 3-(2-naphthyl) alanine
  MOD_RES 8: note = D-lysine
  MOD_RES 12: note = citrulline
  DISULFID 4..13: note = may be linked by disulfide bond between Cys
RRACYRKKPYERCR

SEQ ID NO: 22 - protein, 14 aa, synthetic construct
  REGION 1..14: note = synthetic peptide
  MOD_RES 3: note = 3-(2-naphthyl) alanine
  MOD_RES 8: note = D-lysine
  MOD_RES 12: note = citrulline
  DISULFID 4..13: note = may be linked by disulfide bond between Cys
RRACYRKKPYRRCE

SEQ ID NO: 23 - protein, 14 aa, synthetic construct
  REGION 1..14: note = synthetic peptide
  MOD_RES 3: note = 3-(2-naphthyl) alanine
  MOD_RES 6: note = citrulline
  MOD_RES 8: note = D-glutamic acid
  MOD_RES 12: note = citrulline
  MOD_RES 14: note = C' amidated
  DISULFID 4..13: note = may be linked by disulfide bond between Cys
RRACYRKEPYRRCR

SEQ ID NO: 24 - protein, 14 aa, synthetic construct
  REGION 1..14: note = synthetic peptide
  MOD_RES 3: note = 3-(2-naphthyl) alanine
  MOD_RES 6: note = D-glutamic acid
  MOD_RES 8: note = D-citrulline
  MOD_RES 12: note = citrulline
  MOD_RES 14: note = c' amidated
  DISULFID 4..13: note = may be linked by disulfide bond between Cys
RRACYEKRPYRRCR

SEQ ID NO: 25 - protein, 14 aa, synthetic construct
  REGION 1..14: note = synthetic peptide
  MOD_RES 3: note = 3-(2-naphthyl) alanine
  MOD_RES 6: note = D-glutamic acid
  MOD_RES 8: note = D-glutamic acid
  MOD_RES 12: note = citrulline
  MOD_RES 14: note = c' amidated
  DISULFID 4..13: note = may be linked by disulfide bond between Cys
RRACYEKEPYRRCR

SEQ ID NO: 26 - protein, 14 aa, synthetic construct
  REGION 1..14: note = synthetic peptide
  MOD_RES 1: note = D-glutamic acid
  MOD_RES 3: note = 3-(2-naphthyl) alanine
  MOD_RES 6: note = citrulline
  MOD_RES 8: note = D-glutamic acid
  MOD_RES 12: note = citrulline
  MOD_RES 14: note = c' amidated
  DISULFID 4..13: note = may be linked by disulfide bond between Cys
ERACYRKEPYRRCR

SEQ ID NO: 27 - protein, 14 aa, synthetic construct
  REGION 1..14: note = synthetic peptide
  MOD_RES 3: note = 3-(2-naphthyl) alanine
  MOD_RES 6: note = citrulline
  MOD_RES 8: note = D-glutamic acid
  MOD_RES 10: note = D-glutamic acid
  MOD_RES 12: note = citrulline
  MOD_RES 14: note = c' amidated
  DISULFID 4..13: note = may be linked by disulfide bond between Cys
RRACYRKEPERRCR

SEQ ID NO: 28 - protein, 14 aa, synthetic construct
  REGION 1..14: note = synthetic peptide
  MOD_RES 3: note = 3-(2-naphthyl) alanine
  MOD_RES 6: note = citrulline
  MOD_RES 8: note = D-glutamic acid
  MOD_RES 12: note = D-glutamic acid
  MOD_RES 14: note = c' amidated
  DISULFID 4..13: note = may be linked by disulfide bond between Cys
RRACYRKEPYRECR

SEQ ID NO: 29 - protein, 14 aa, synthetic construct
  REGION 1..14: note = synthetic peptide
  MOD_RES 1: note = N' ACETYLATED D-glutamic acid
  MOD_RES 3: note = 3-(2-naphthyl) alanine
  MOD_RES 6: note = citrulline
  MOD_RES 8: note = D-glutamic acid
  MOD_RES 12: note = citrulline
  MOD_RES 14: note = c' amidated
  DISULFID 4..13: note = may be linked by disulfide bond between Cys
ERACYRKEPYRRCR

SEQ ID NO: 30 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 6: note = citrulline
  MOD_RES 8: note = D-glutamic acid
  MOD_RES 10: note = D-glutamic acid
  MOD_RES 12: note = citrulline
  MOD_RES 14: note = c' amidated
  DISULFID 4..13: note = may be linked by disulfide bond between Cys
RRACYRKEPERRCR

SEQ ID NO: 31 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 6: note = citrulline
  MOD_RES 8: note = D-glutamic acid
  MOD_RES 12: note = D-glutamic acid
  MOD_RES 14: note = c' amidated
  DISULFID 4..13: note = may be linked by disulfide bond between Cys
RRACYRKEPYRECR

SEQ ID NO: 32 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 6: note = citrulline
  MOD_RES 8: note = D-glutamic acid
  MOD_RES 12: note = citrulline
  MOD_RES 14: note = c' amidated
  DISULFID 4..13: note = may be linked by disulfide bond between Cys
RRACYRKEPYRRCR

SEQ ID NO: 33 - protein, 14 aa, synthetic construct
  REGION 1..14: note = synthetic peptide
  MOD_RES 1: note = Guanyl-arginine
  MOD_RES 3: note = 3-(2-naphthyl) alanine
  MOD_RES 6: note = citrulline
  MOD_RES 8: note = D-glutamic acid
  MOD_RES 12: note = citrulline
  MOD_RES 14: note = c' amidated
  DISULFID 4..13: note = may be linked by disulfide bond between Cys
RRACYRKEPYRRCR

SEQ ID NO: 34 - protein, 14 aa, synthetic construct
  REGION 1..14: note = synthetic peptide
  MOD_RES 1: note = Tetramethylguanyl-arginine
  MOD_RES 3: note = 3-(2-naphthyl) alanine
  MOD_RES 6: note = citrulline
  MOD_RES 8: note = D-glutamic acid
  MOD_RES 12: note = citrulline
  MOD_RES 14: note = c' amidated
  DISULFID 4..13: note = may be linked by disulfide bond between Cys
RRACYRKEPYRRCR

SEQ ID NO: 35 - protein, 13 aa, synthetic construct
  REGION 1..13: note = synthetic peptide
  MOD_RES 1: note = Tetramethylguanyl-arginine
  MOD_RES 2: note = 3-(2-naphthyl) alanine
  MOD_RES 5: note = citrulline
  MOD_RES 7: note = D-glutamic acid
  MOD_RES 11: note = citrulline
  MOD_RES 13: note = c' amidated
  DISULFID 3..12: note = may be linked by disulfide bond between Cys
RACYRKEPYRRCR

SEQ ID NO: 36 - protein, 14 aa, synthetic construct
  REGION 1..14: note = synthetic peptide
  MOD_RES 1: note = 4-fluorobenzoyl-arginine
  MOD_RES 3: note = 3-(2-naphthyl) alanine
  MOD_RES 6: note = citrulline
  MOD_RES 8: note = D-glutamic acid
  MOD_RES 12: note = citrulline
  MOD_RES 14: note = amidated
  DISULFID 4..13: note = may be linked by disulfide bond between Cys
RRACYRKEPYRRCR

SEQ ID NO: 37 - protein, 14 aa, synthetic construct
  REGION 1..14: note = synthetic peptide
  MOD_RES 1: note = 2-fluorobenzoyl-arginine
  MOD_RES 3: note = 3-(2-naphthyl) alanine
  MOD_RES 6: note = citrulline
  MOD_RES 8: note = D-glutamic acid
  MOD_RES 12: note = citrulline
  MOD_RES 14: note = c' amidated
  DISULFID 4..13: note = may be linked by disulfide bond between Cys
RRACYRKEPYRRCR

SEQ ID NO: 38 - protein, 13 aa, synthetic construct
  REGION 1..13: note = synthetic peptide
  MOD_RES 1: note = 5-aminopentanoyl-arginine
  MOD_RES 2: note = 3-(2-naphthyl) alanine
  MOD_RES 5: note = citrulline
  MOD_RES 7: note = D-glutamic acid
  MOD_RES 11: note = citrulline
  MOD_RES 13: note = c' amidated
  DISULFID 3..12: note = may be linked by disulfide bond between Cys
RACYRKEPYRRCR

SEQ ID NO: 39 - protein, 14 aa, synthetic construct
  REGION 1..14: note = synthetic peptide
  MOD_RES 1: note = 2-desamino-arginyl
  MOD_RES 3: note = 3-(2-naphthyl) alanine
  MOD_RES 6: note = citrulline
  MOD_RES 8: note = D-glutamic acid
  MOD_RES 12: note = citrulline
  MOD_RES 14: note = c' amidated
  DISULFID 3..12: note = may be linked by disulfide bond between Cys
RRACYRKEPYRRCR

SEQ ID NO: 40 - protein, 13 aa, synthetic construct
  REGION 1..13: note = synthetic peptide
  MOD_RES 1: note = Guanyl-arginine
  MOD_RES 2: note = 3-(2-naphthyl) alanine
  MOD_RES 5: note = citrulline
  MOD_RES 7: note = D-glutamic acid
  MOD_RES 11: note = citrulline
  MOD_RES 13: note = c' amidated
  DISULFID 3..12: note = may be linked by disulfide bond between Cys
RACYRKEPYRRCR

SEQ ID NO: 41 - protein, 13 aa, synthetic construct
  REGION 1..13: note = synthetic peptide
  MOD_RES 1: note = Succinyl-arginine
  MOD_RES 2: note = 3-(2-naphthyl) alanine
  MOD_RES 5: note = citrulline
  MOD_RES 7: note = D-glutamic acid
  MOD_RES 11: note = citrulline
  MOD_RES 13: note = c' amidated
  DISULFID 3..12: note = may be linked by disulfide bond between Cys
RACYRKEPYRRCR

SEQ ID NO: 42 - protein, 13 aa, synthetic construct
  REGION 1..13: note = synthetic peptide
  MOD_RES 1: note = Glutaryl-arginine
  MOD_RES 2: note = 3-(2-naphthyl) alanine
  MOD_RES 5: note = citrulline
  MOD_RES 7: note = D-glutamic acid
  MOD_RES 11: note = citrulline
  MOD_RES 13: note = c' amidated
  DISULFID 3..12: note = may be linked by disulfide bond between Cys
RACYRKEPYRRCR

SEQ ID NO: 43 - protein, 13 aa, synthetic construct
  REGION 1..13: note = synthetic peptide
  MOD_RES 1: note = desaminoTMG-APA conjugated
  MOD_RES 2: note = 3-(2-naphthyl) alanine
  MOD_RES 5: note = citrulline
  MOD_RES 7: note = D-glutamic acid
  MOD_RES 11: note = citrulline
  MOD_RES 13: note = c' amidated
  DISULFID 3..12: note = may be linked by disulfide bond between Cys
RACYRKEPYRRCR

SEQ ID NO: 44 - protein, 13 aa, synthetic construct
  REGION 1..13: note = synthetic peptide
  MOD_RES 1: note = R-CH2 -conjugated ( formula (V) in the specification)
  MOD_RES 3: note = 3-(2-naphthyl) alanine
  MOD_RES 5: note = citrulline
  MOD_RES 7: note = D-glutamic acid
  MOD_RES 11: note = citrulline
  MOD_RES 13: note = c' amidated
  DISULFID 3..12: note = may be linked by disulfide bond between Cys
RACYRKEPYRRCR

SEQ ID NO: 45 - protein, 13 aa, synthetic construct
  REGION 1..13: note = synthetic peptide
  MOD_RES 2: note = 3-(2-naphthyl) alanine
  MOD_RES 5: note = citrulline
  MOD_RES 7: note = D-glutamic acid
  MOD_RES 11: note = citrulline
  MOD_RES 13: note = c' amidated
  DISULFID 3..12: note = may be linked by disulfide bond between Cys
RACYRKEPYRRCR

SEQ ID NO: 46 - protein, 14 aa, synthetic construct
  REGION 1..14: note = synthetic peptide
  MOD_RES 1: note = tetramethylguanyl-arginine
  MOD_RES 3: note = 3-(2-naphthyl) alanine
  MOD_RES 6: note = citrulline
  MOD_RES 8: note = D-citrulline
  MOD_RES 12: note = citrulline
  MOD_RES 14: note = C' AMIDATED
  DISULFID 4..13: note = may be linked by disulfide bond between Cys
RRACYRKRPYRRCR

SEQ ID NO: 47 - protein, 14 aa, synthetic construct
  REGION 1..14: note = synthetic peptide
  MOD_RES 1: note = 6-aminohexanoyl-arginine
  MOD_RES 3: note = 3-(2-naphthyl) alanine
  MOD_RES 6: note = citrulline
  MOD_RES 8: note = D-citrulline
  MOD_RES 12: note = citrulline
  MOD_RES 14: note = C' AMIDATED
  DISULFID 4..13: note = may be linked by disulfide bond between Cys
RRACYRKRPYRRCR

SEQ ID NO: 48 - protein, 14 aa, synthetic construct
  REGION 1..14: note = synthetic peptide
  MOD_RES 1: note = 6-aminohexanoyl-arginine
  MOD_RES 3: note = 3-(2-naphthyl) alanine
  MOD_RES 8: note = D-lysine
  MOD_RES 12: note = citrulline
  DISULFID 4..13: note = may be linked by disulfide bond between Cys
RRACYRKKPYRRCR

SEQ ID NO: 49 - protein, 14 aa, synthetic construct
  REGION 1..14: note = synthetic peptide
  MOD_RES 3: note = 3-(2-naphthyl) alanine
  MOD_RES 6: note = citrulline
  MOD_RES 8: note = D-lysine
  MOD_RES 12: note = citrulline
  MOD_RES 14: note = C' amidated
  DISULFID 4..13: note = may be linked by disulfide bond between Cys
RRACYRRKPYRRCR

SEQ ID NO: 50 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 6: note = citrulline
  MOD_RES 8: note = D-lysine
  MOD_RES 12: note = citrulline
  MOD_RES 14: note = C' amidated
  DISULFID 4..13: note = may be linked by disulfide bond between Cys
RRACYRRKPYRRCR

SEQ ID NO: 51 - protein, 14 aa, synthetic construct
  REGION 1..14: note = SYNTHETIC PEPTIDE
  MOD_RES 1: note = N' ACETYLATED
  MOD_RES 3: note = 3-(2-naphthyl) alanine
  MOD_RES 6: note = citrulline
  MOD_RES 8: note = D-lysine
  MOD_RES 12: note = citrulline
  MOD_RES 14: note = C' AMIDATED
  DISULFID 4..13: note = may be linked by disulfide bond between Cys
RRACYRKKPYRRCR

SEQ ID NO: 52 - protein, 14 aa, synthetic construct
  REGION 1..14: note = synthetic peptide
  MOD_RES 1: note = N' ACETYLATED
  MOD_RES 3: note = 3-(2-naphthyl) alanine
  MOD_RES 8: note = D-citrulline
  MOD_RES 12: note = citrulline
  MOD_RES 14: note = C' AMIDATED
  DISULFID 4..13: note = may be linked by disulfide bond between Cys
RRACYRKRPYRRCR

SEQ ID NO: 53 - protein, 14 aa, synthetic construct
  REGION 1..14: note = synthetic peptide
  MOD_RES 1: note = 4-fluorobenzoyl-arginine
  MOD_RES 3: note = 3-((2-naphthyl) alanine
  MOD_RES 6: note = citrulline
  MOD_RES 8: note = D-glutamic acid
  MOD_RES 12: note = citrulline
  SITE 14: note = MISC_FEATURE - derivatization by a NH-methyl group
  DISULFID 4..13: note = may be linked by disulfide bond between Cys
RRACYRKEPYRRCR

SEQ ID NO: 54 - protein, 14 aa, synthetic construct
  REGION 1..14: note = synthetic peptide
  MOD_RES 1: note = 4-fluorobenzoyl-arginine
  MOD_RES 3: note = 3-((2-naphthyl) alanine
  MOD_RES 6: note = citrulline
  MOD_RES 8: note = D-glutamic acid
  MOD_RES 12: note = citrulline
  SITE 14: note = MISC_FEATURE - derivatization by a NH-ethyl group
  DISULFID 4..13: note = may be linked by disulfide bond between Cys
RRACYRKEPYRRCR

SEQ ID NO: 55 - protein, 14 aa, synthetic construct
  REGION 1..14: note = synthetic peptide
  MOD_RES 1: note = 4-fluorobenzoyl-arginine
  MOD_RES 3: note = 3-((2-naphthyl) alanine
  MOD_RES 6: note = citrulline
  MOD_RES 8: note = D-glutamic acid
  MOD_RES 12: note = citrulline
  SITE 14: note = MISC_FEATURE - derivatization by NH-isopropyl
  DISULFID 4..13: note = may be linked by disulfide bond between Cys
RRACYRKEPYRRCR

SEQ ID NO: 56 - protein, 14 aa, synthetic construct
  REGION 1..14: note = synthetic peptide
  MOD_RES 1: note = 4-fluorobenzoyl-arginine
  MOD_RES 3: note = 3-((2-naphthyl) alanine
  MOD_RES 6: note = citrulline
  MOD_RES 8: note = D-glutamic acid
  MOD_RES 12: note = citrulline
  SITE 14: note = MISC_FEATURE - derivatization with a tyramine residue
  DISULFID 4..13: note = may be linked by disulfide bond between Cys
RRACYRKEPYRRCR

SEQ ID NO: 57 - protein, 14 aa, synthetic construct
  REGION 1..14: note = synthetic peptide
  MOD_RES 3: note = 3-((2-naphthyl) alanine
  MOD_RES 8: note = D-lysine
  MOD_RES 12: note = citrulline
  DISULFID 4..13: note = may be linked by disulfide bond between Cys
ARACYRKKPYRRCR

SEQ ID NO: 58 - protein, 14 aa, synthetic construct
  REGION 1..14: note = synthetic peptide
  MOD_RES 3: note = 3-((2-naphthyl) alanine
  MOD_RES 8: note = D-lysine
  MOD_RES 12: note = citrulline
  DISULFID 4..13: note = may be linked by disulfide bond between Cys
RRACYAKKPYRRCR

SEQ ID NO: 59 - protein, 14 aa, synthetic construct
  REGION 1..14: note = synthetic peptide
  MOD_RES 3: note = 3-((2-naphthyl) alanine
  MOD_RES 8: note = D-lysine
  MOD_RES 12: note = citrulline
  DISULFID 4..13: note = may be linked by disulfide bond between Cys
RRACYRAKPYRRCR

SEQ ID NO: 60 - protein, 14 aa, synthetic construct
  REGION 1..14: note = synthetic peptide
  MOD_RES 3: note = 3-((2-naphthyl) alanine
  MOD_RES 8: note = D-alanine
  MOD_RES 12: note = citrulline
  DISULFID 4..13: note = may be linked by disulfide bond between Cys
RRACYRKAPYRRCR

SEQ ID NO: 61 - protein, 14 aa, synthetic construct
  REGION 1..14: note = synthetic peptide
  MOD_RES 3: note = 3-((2-naphthyl) alanine
  MOD_RES 8: note = D-lysine
  MOD_RES 12: note = citrulline
  DISULFID 4..13: note = may be linked by disulfide bond between Cys
RRACYRKKAYRRCR

SEQ ID NO: 62 - protein, 14 aa, synthetic construct
  REGION 1..14: note = synthetic peptide
  MOD_RES 3: note = 3-((2-naphthyl) alanine
  MOD_RES 8: note = D-lysine
  MOD_RES 12: note = citrulline
  DISULFID 4..13: note = may be linked by disulfide bond between Cys
RRACYRKKPARRCR

SEQ ID NO: 63 - protein, 14 aa, synthetic construct
  REGION 1..14: note = synthetic peptide
  MOD_RES 3: note = 3-((2-naphthyl) alanine
  MOD_RES 8: note = D-lysine
  MOD_RES 12: note = citrulline
  DISULFID 4..13: note = may be linked by disulfide bond between Cys
RRACYRKKPYARCR

SEQ ID NO: 64 - protein, 14 aa, synthetic construct
  REGION 1..14: note = synthetic peptide
  MOD_RES 1: note = citrulline
  MOD_RES 3: note = 3-((2-naphthyl) alanine
  MOD_RES 8: note = D-lysine
  MOD_RES 12: note = citrulline
  DISULFID 4..13: note = may be linked by disulfide bond between Cys
RRACYRKKPYRRCR

SEQ ID NO: 65 - protein, 14 aa, synthetic construct
  REGION 1..14: note = synthetic peptide
  MOD_RES 3: note = 3-((2-naphthyl) alanine
  MOD_RES 6: note = citrulline
  MOD_RES 8: note = D-lysine
  MOD_RES 12: note = citrulline
  DISULFID 4..13: note = may be linked by disulfide bond between Cys
RRACYRKKPYRRCR

SEQ ID NO: 66 - protein, 14 aa, synthetic construct
  REGION 1..14: note = synthetic peptide
  MOD_RES 3: note = 3-((2-naphthyl) alanine
  MOD_RES 6: note = citrulline
  MOD_RES 8: note = D-lysine
  MOD_RES 12: note = citrulline
  MOD_RES 14: note = C' AMIDATED
  DISULFID 4..13: note = may be linked by disulfide bond between Cys
RRACYRKKPYRRCR

SEQ ID NO: 67 - protein, 14 aa, synthetic construct
  REGION 1..14: note = synthetic peptide
  MOD_RES 3: note = 3-((2-naphthyl) alanine
  MOD_RES 7: note = citrulline
  MOD_RES 8: note = D-lysine
  MOD_RES 12: note = citrulline
  DISULFID 4..13: note = may be linked by disulfide bond between Cys
RRACYRRKPYRRCR

SEQ ID NO: 68 - protein, 14 aa, synthetic construct
  REGION 1..14: note = synthetic peptide
  MOD_RES 3: note = 3-((2-naphthyl) alanine
  MOD_RES 6: note = citrulline
  MOD_RES 8: note = D-citrulline
  MOD_RES 12: note = citrulline
  MOD_RES 14: note = C' AMIDATED
  DISULFID 4..13: note = may be linked by disulfide bond between Cys
RRACYRKRPYRRCR

SEQ ID NO: 69 - protein, 14 aa, synthetic construct
  REGION 1..14: note = synthetic peptide
  MOD_RES 3: note = 3-((2-naphthyl) alanine
  MOD_RES 8: note = D-lysine
  MOD_RES 12: note = citrulline
  DISULFID 4..13: note = may be linked by disulfide bond between Cys
RRACYRKKPYRRCR

SEQ ID NO: 70 - protein, 14 aa, synthetic construct
  REGION 1..14: note = synthetic peptide
  MOD_RES 3: note = 3-((2-naphthyl) alanine
  MOD_RES 6: note = citrulline
  MOD_RES 8: note = D-citrulline
  MOD_RES 12: note = citrulline
  DISULFID 4..13: note = may be linked by disulfide bond between Cys
RRACYRKRPYRRCR

SEQ ID NO: 71 - protein, 14 aa, synthetic construct
  REGION 1..14: note = synthetic peptide
  MOD_RES 3: note = 3-((2-naphthyl) alanine
  MOD_RES 8: note = D-citrulline
  MOD_RES 12: note = citrulline
  DISULFID 4..13: note = may be linked by disulfide bond between Cys
RRACYRKRPYRRCR

SEQ ID NO: 72 - protein, 14 aa, synthetic construct
  REGION 1..14: note = synthetic peptide
  MOD_RES 1: note = citrulline
  MOD_RES 3: note = 3-((2-naphthyl) alanine
  MOD_RES 8: note = D-citrulline
  MOD_RES 12: note = citrulline
  MOD_RES 14: note = C' Amidated
  DISULFID 4..13: note = may be linked by disulfide bond between Cys
RRACYRKRPYRRCR

(19) United States
(12) Patent Application Publication
(10) Pub. No.: US 2026/0201040 A1
(43) Pub. Date: Jul. 16, 2026

(54) TREATMENT OF SICKLE CELL DISEASE

(71) Applicants: BioLineRx Ltd., ModiIn (IL); The Washington University, St. Louis, MO (US)

(72) Inventors: John F. DIPERSIO, St. Louis, MO (US); Abi VAINSTEIN-HARAS, Herzliya (IL); Peter RUMINSKI, St. Louis, MO (US); Michael RETTIG, St. Louis, MO (US)

(73) Assignees: BioLineRx Ltd., ModiIn (IL); The Washington University, St. Louis, MO (US)

(21) Appl. No.: 19/137,719

(22) PCT Filed: Dec. 11, 2023
(86) PCT No.: PCT/IL2023/051261

§ 371 Date: Jun. 11, 2025

Related U.S. Application Data
(60) Provisional application No. 63/431,705, filed on Dec. 11, 2022.

(51) Int. Cl. C07K 16/28; A61K 38/12; A61K 39/00; A61K 48/00

(52) U.S. Cl. CPC C07K 16/2839; A61K 38/12; A61K 48/005; A61K 2039/54; A61K 2039/545

(57) ABSTRACT

A method of mobilizing CD34+ cells in a subject suffering from sickle cell disease (SCD) is provided. The method comprising administering to the subject a therapeutically effective amount of a CXCR4 inhibitory peptide as set forth in SEQ ID NO: 1 and a therapeutically effective amount of Natalizumab, wherein said Natalizumab and said CXCR4 inhibitory peptide are administered sequentially. Also provided is use of the immobilized cells in gene therapy for subsequent treatment of SCD.

### RELATED APPLICATION/S

This application claims the benefit of priority of U.S. Provisional Patent Application No. 63/431,705 filed on 11 Dec. 2022, the contents of which are incorporated herein by reference in their entirety.

### SEQUENCE LISTING STATEMENT

The XML file, entitled 97965 Sequence Listing.xml, created on 11 Dec. 2023, comprising 217,088 bytes, submitted concurrently with the filing of this application is incorporated herein by reference.

### FIELD AND BACKGROUND OF THE INVENTION

The present invention, in some embodiments thereof, relates to treatment of sickle cell disease.

Sickle cell disease (SCD) is one of the most common genetic diseases globally. Clinical manifestations of SCD commonly include anemia, pain, vaso-occlusive events and cumulative end organ damage leading to significant morbidity and mortality. Historically, allogeneic hematopoietic cell transplantation (HCT) represented the only curative therapy for SCD, but is associated with significant HCT-related morbidity and mortality which limit use (PMID: 8663884, PMID: 20007560).

Autologous hematopoietic stem cell (HSC)-based gene therapies now offer curative potential with decreased toxicity (PMID: 28249145). However, effective HSC-based gene therapy depends on collection of sufficient HSCs (ideally ~10-15×10⁶ CD34+ cells/kg), typically from peripheral blood (PB) (PMID: 33956057). G-CSF and CXCR4 inhibition (CXCR4i) with plerixafor are the most widely used mobilization strategies (PMID: 19363221, PMID: 19720922). However, G-CSF is associated with fatal vaso-occlusive events in SCD (PMID: 9734950, PMID: 11368061, PMID: 19513902). Meanwhile, short-acting CXCR4i with plerixafor alone does not reliably yield optimal HSC numbers for gene therapy applications (PMID: 30282642, PMID: 29419425, PMID: 29472357). Therefore, developing novel HSC mobilization regimens to rapidly and reliably mobilize optimal CD34+ HSCs for gene therapy in SCD represents an unmet need.

Additional background art includes:

### SUMMARY OF THE INVENTION

According to an aspect of some embodiments of the present invention there is provided a method of mobilizing CD34+ cells in a subject suffering from sickle cell disease (SCD), the method comprising administering to the subject a therapeutically effective amount of a CXCR4 inhibitory peptide as set forth in SEQ ID NO: 1 and a therapeutically effective amount of Natalizumab, wherein the Natalizumab and the CXCR4 inhibitory peptide are administered sequentially.

According to an aspect of some embodiments of the present invention there is provided a combination of agents for use in treating sickle cell disease (SCD) the combination comprising a therapeutically effective amount of a CXCR4 inhibitory peptide as set forth in SEQ ID NO: 1 and a therapeutically effective amount of Natalizumab, wherein the Natalizumab and the CXCR4 inhibitory peptide are administered sequentially.

According to some embodiments of the invention, the Natalizumab is administered intravenously (i.v.).

According to some embodiments of the invention, the peptide is formulated as Motixafortide.

According to some embodiments of the invention, the peptide is administered subcutaneously (S.C.).

According to some embodiments of the invention, the peptide is administered at a dose of 0.1 to 10 mg/kg of body weight.

According to some embodiments of the invention, the peptide is administered at a dose of 1-2 mg/kg of body weight.

According to some embodiments of the invention, the peptide is administered at a dose of 1.25 mg/kg of body weight.

According to some embodiments of the invention, the Natalizumab is administered intravenously (i.v.).

According to some embodiments of the invention, the Natalizumab is administered at a dose of 100-1000 mg.

According to some embodiments of the invention, the Natalizumab is administered at a dose of 300 mg.

According to some embodiments of the invention, the Natalizumab is administered prior to the peptide.

According to some embodiments of the invention, the peptide is administered at a time point not exceeding 24 hours following administration of the Natalizumab.

According to some embodiments of the invention, the peptide is administered 24 hours following administration of the Natalizumab.

According to some embodiments of the invention, the method comprises performing apheresis to retrieve the CD34+ cells from peripheral blood of the subject following administering the agents.

According to an aspect of some embodiments of the present invention there is provided a population of mobilized cells obtainable according to the methods described herein.

According to an aspect of some embodiments of the present invention there is provided a method of treating sickle cells disease (SCD), the method comprising:
(a) mobilizing CD34+ cells in a subject suffering from sickle cell disease (SCD) as described herein;
(b) performing apheresis to retrieve the CD34+ cells from peripheral blood of the subject; and
(c) treating the CD34+ cells to obtain red blood cells with a healthy phenotype.

According to some embodiments of the invention, the treating is by genome editing.

According to some embodiments of the invention, the healthy phenotype comprises expression of fetal hemoglobin (HbF; hemoglobin F).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of the study design. The study compares treatments of Motixafortide (M) to M+N on a study group.

FIG. 2 is a scheme describing the lineages produced from CD34+ cells. HSC (heamtopoeitic stem cell—a multi potent cell—can differentiate to all lineages of tissue, with self renewal. MPP, a Multi-Potent, Progenitor, a cell population that has lost the self-renewal capacity of HSC. LMPP, lymphoid-primed multipotent progenitor, has virtually exclusively a lymphoid progenitor with very low myeloid output (3%). CMP (common myeloid progenitor) a oligopotent—can differentiate to several tissues/organ but not all. MEP: Megakaryocyte/erythrocyte progenitor, a lineage-restricted progenitors, evolved from CMP. GMP: granulocyte/macrophage progenitor, a lineage-restricted progenitors, evolved from CMP and LMPP. CLP: common lymphoid progenitor, oligopotent—can differentiate to several tissues/organ but not all.

### DESCRIPTION OF SPECIFIC EMBODIMENTS OF THE INVENTION

The present invention, in some embodiments thereof, relates to treatment of sickle cell disease (SCD).

Developing novel HSC mobilization regimens to rapidly and reliably mobilize optimal CD34+ HSCs for gene therapy in SCD represents an unmet need.

Whilst conceiving embodiments of the invention, the present inventors envisaged that combining VLA4 inhibition by Natalizumab with CXCR4 inhibition by Motixafortide synergistically increase HSC mobilization.

Thus, according to an aspect of the invention there is provided a method of mobilizing CD34+ cells in a subject suffering from sickle cell disease (SCD), the method comprising administering to the subject a therapeutically effective amount of a CXCR4 inhibitory peptide as set forth in SEQ ID NO: 1 and a therapeutically effective amount of Natalizumab, wherein said Natalizumab and said CXCR4 inhibitory peptide are administered sequentially.

According to an aspect of the invention there is provided a combination of agents for use in treating sickle cell disease (SCD) said combination comprising a therapeutically effective amount of a CXCR4 inhibitory peptide as set forth in SEQ ID NO: 1 and a therapeutically effective amount of Natalizumab, wherein said Natalizumab and said CXCR4 inhibitory peptide are administered sequentially.

As used herein “sickle cell disease” abbreviated as SCD refers to any form of these inherited red blood cell disorders. Red blood cells contain hemoglobin, a protein that carries oxygen. Healthy red blood cells are round, and they move through small blood vessels to carry oxygen to all parts of the body. In someone who has SCD, the hemoglobin is abnormal, which causes the red blood cells to become hard and sticky and look like a C-shaped farm tool called a “sickle.” Specific forms of the disease include HbSS, HBSc, HbS and HbSD, HbAS, HbSE, and HbSO, each of which is contemplated herein and is considered an independent embodiment. The disease beta thalassemia is included also considered under this term (SCD), according to a specific embodiment of the invention.

According to a specific embodiment, the subject is diagnosed with SCD.

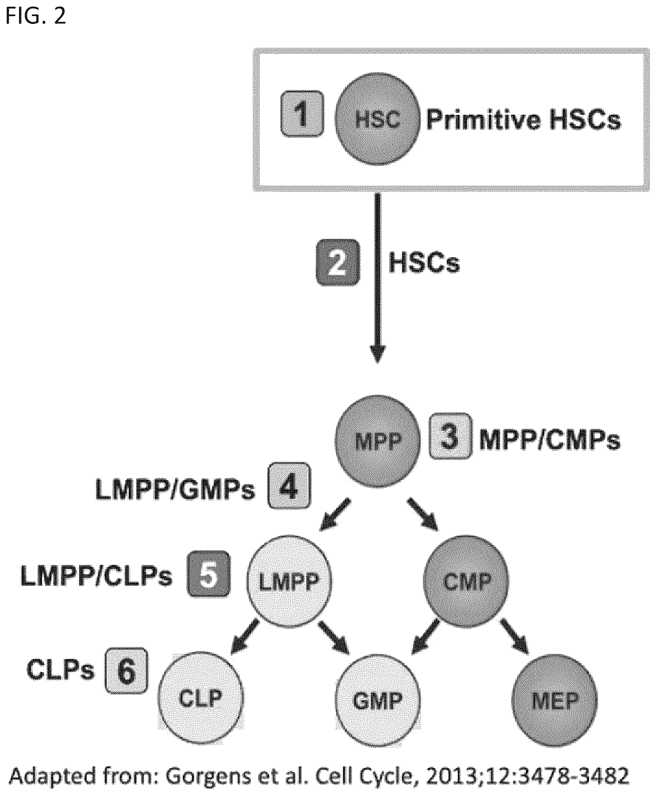

As used herein CD34+ cells refer to the hempatopoietic stem cells in human (HSCs) that can develop to red blood cells and can be the subject for genetic manipulation and research (e.g., immunophenotyping, transcription assays, e.g., scRNA seq and mFACS, see FIG. 2). CD34+ cells can be assayed using fluorescence activated cell sorting (FACS) and thus their presence can be assessed in a sample using this technique. In general, CD34+ cells are present only in low levels in the blood, but are present in large numbers in bone marrow.

According to specific embodiments, the CXCR4-inhibitory peptide of the present invention is 4F-benzoyl-TN14003 (SEQ ID NO: 1), however analogs and derivatives are also contemplated. These are structurally and functionally related to the peptides disclosed in patent applications WO 2002/020561 and WO 2004/020462, also known as “T-140 analogs”, as detailed hereinbelow.

In various particular embodiments, the T-140 analog or derivative has an amino acid sequence as set forth in the following formula (I) or a salt thereof:

1 2 3 4 5 6 7 8 9 10 11 12 13 14
A₁-A₂-A₃-Cys-Tyr-A₄-A₅-A₆-A₇-A₈-A₉-A₁₀-Cys-A₁₁ (I)

wherein:
A₁ is an arginine, lysine, ornithine, citrulline, alanine or glutamic acid residue or a N-α-substituted derivative of these amino acids, or A₁ is absent;
A₂ represents an arginine or glutamic acid residue if A₁ is present, or A₂ represents an arginine or glutamic acid residue or a N-α-substituted derivative of these amino acids if A₁ is absent;
A₃ represents an aromatic amino acid residue;
A₄, A₅ and A₉ each independently represents an arginine, lysine, ornithine, citrulline, alanine or glutamic acid residue;
A₆ represents a proline, glycine, ornithine, lysine, alanine, citrulline, arginine or glutamic acid residue;
A₇ represents a proline, glycine, ornithine, lysine, alanine, citrulline or arginine residue;
A₈ represents a tyrosine, phenylalanine, alanine, naphthylalanine, citrulline or glutamic acid residue;
A₁₀ represents a citrulline, glutamic acid, arginine or lysine residue;
A₁₁ represents an arginine, glutamic acid, lysine or citrulline residue wherein the C-terminal carboxyl may be derivatized;
and the cysteine residue of the 4-position or the 13-position can form a disulfide bond, and the amino acids can be of either L or D form.

Exemplary peptides according to formula (I) are peptides having an amino acid sequence as set forth in any one of SEQ ID NOS: 1-72, as presented in Table 1 hereinbelow.

TABLE 1
T-140 and currently preferred T-140 analogs

| | SEQ | |
|---|---|---|
| Analog | ID NO: | Amino acid sequence |
| 4F-benzoyl- | 1 | 4F-benzoyl-Arg-Arg-Nal-Cys-Tyr-Cit-Lys-DLys-Pro-Tyr-Arg-Cit-Cys- |
| TN14003 | | Arg-NH₂ |
| AcTC14003 | 2 | Ac-Arg-Arg-Nal-Cys-Tyr-Cit-Lys-DLys-Pro-Tyr-Arg-Cit-Cys-Arg-OH |
| AcTC14005 | 3 | Ac-Arg-Arg-Nal-Cys-Tyr-Arg-Lys-DCit-Pro-Tyr-Arg-Cit-Cys-Arg-OH |
| AcTC14011 | 4 | Ac-Arg-Arg-Nal-Cys-Tyr-Cit-Lys-DCit-Pro-Tyr-Arg-Cit-Cys-Arg-OH |
| AcTC14013 | 5 | Ac-Arg-Arg-Nal-Cys-Tyr-Cit-Lys-DLys-Pro-Tyr-Cit-Cit-Cys-Arg-OH |
| AcTC14015 | 6 | Ac-Cit-Arg-Nal-Cys-Tyr-Cit-Lys-DLys-Pro-Tyr-Arg-Cit-Cys-Arg-OH |
| AcTC14017 | 7 | Ac-Cit-Arg-Nal-Cys-Tyr-Arg-Lys-DCit-Pro-Tyr-Arg-Cit-Cys-Arg-OH |
| AcTC14019 | 8 | Ac-Arg-Arg-Nal-Cys-Tyr-Arg-Lys-DCit-Pro-Tyr-Cit-Cit-Cys-Arg-OH |
| AcTC14021 | 9 | Ac-Cit-Arg-Nal-Cys-Tyr-Arg-Lys-DLys-Pro-Tyr-Cit-Cit-Cys-Arg-OH |
| AcTC14012 | 10 | Ac-Arg-Arg-Nal-Cys-Tyr-Cit-Lys-DCit-Pro-Tyr-Arg-Cit-Cys-Arg-NH₂ |
| AcTC14014 | 11 | Ac-Arg-Arg-Nal-Cys-Tyr-Cit-Lys-DLys-Pro-Tyr-Cit-Cit-Cys-Arg-NH₂ |
| AcTC14016 | 12 | Ac-Cit-Arg-Nal-Cys-Tyr-Cit-Lys-DLys-Pro-Tyr-Arg-Cit-Cys-Arg-NH₂ |
| AcTC14018 | 13 | Ac-Cit-Arg-Nal-Cys-Tyr-Arg-Lys-DCit-Pro-Tyr-Arg-Cit-Cys-Arg-NH₂ |
| AcTC14020 | 14 | Ac-Arg-Arg-Nal-Cys-Tyr-Arg-Lys-DCit-Pro-Tyr-Cit-Cit-Cys-Arg-NH₂ |
| AcTC14022 | 15 | Ac-Cit-Arg-Nal-Cys-Tyr-Arg-Lys-DLys-Pro-Tyr-Cit-Cit-Cys-Arg-NH₂ |
| TE14001 | 16 | H-DGlu-Arg-Nal-Cys-Tyr-Arg-Lys-DLys-Pro-Tyr-Arg-Cit-Cys-Arg-OH |
| TE14002 | 17 | H-Arg-Glu-Nal-Cys-Tyr-Arg-Lys-DLys-Pro-Tyr-Arg-Cit-Cys-Arg-OH |
| TE14003 | 18 | H-Arg-Arg-Nal-Cys-Tyr-Glu-Lys-DLys-Pro-Tyr-Arg-Cit-Cys-Arg-OH |
| TE14004 | 19 | H-Arg-Arg-Nal-Cys-Tyr-Arg-Glu-DLys-Pro-Tyr-Arg-Cit-Cys-Arg-OH |
| TE14005 | 20 | H-Arg-Arg-Nal-Cys-Tyr-Arg-Lys-DGlu-Pro-Tyr-Arg-Cit-Cys-Arg-OH |
| TE14006 | 21 | H-Arg-Arg-Nal-Cys-Tyr-Arg-Lys-DLys-Pro-Tyr-Glu-Cit-Cys-Arg-OH |
| TE14007 | 22 | H-Arg-Arg-Nal-Cys-Tyr-Arg-Lys-DLys-Pro-Tyr-Arg-Cit-Cys-Glu-OH |
| TE14011 | 23 | H-Arg-Arg-Nal-Cys-Tyr-Cit-Lys-DGlu-Pro-Tyr-Arg-Cit-Cys-Arg-NH₂ |
| TE14012 | 24 | H-Arg-Arg-Nal-Cys-Tyr-DGlu-Lys-DCit-Pro-Tyr-Arg-Cit-Cys-Arg-NH₂ |
| TE14013 | 25 | H-Arg-Arg-Nal-Cys-Tyr-DGlu-Lys-DGlu-Pro-Tyr-Arg-Cit-Cys-Arg-NH₂ |
| TE14014 | 26 | H-DGlu-Arg-Nal-Cys-Tyr-Cit-Lys-DGlu-Pro-Tyr-Arg-Cit-Cys-Arg-NH₂ |
| TE14015 | 27 | H-Arg-Arg-Nal-Cys-Tyr-Cit-Lys-DGlu-Pro-DGlu-Arg-Cit-Cys-Arg-NH₂ |
| TE14016 | 28 | H-Arg-Arg-Nal-Cys-Tyr-Cit-Lys-DGlu-Pro-Tyr-Arg-DGlu-Cys-Arg-NH₂ |
| AcTE14014 | 29 | Ac-DGlu-Arg-Nal-Cys-Tyr-Cit-Lys-DGlu-Pro-Tyr-Arg-Cit-Cys-Arg-NH₂ |
| AcTE14015 | 30 | Ac-Arg-Arg-Nal-Cys-Tyr-Cit-Lys-DGlu-Pro-DGlu-Arg-Cit-Cys-Arg-NH₂ |
| AcTE14016 | 31 | Ac-Arg-Arg-Nal-Cys-Tyr-Cit-Lys-DGlu-Pro-Tyr-Arg-DGlu-Cys-Arg-NH₂ |
| TF1: | 32 | Ac-Arg-Arg-Nal-Cys-Tyr-Cit-Lys-DGlu-Pro-Tyr-Arg-Cit-Cys-Arg-NH₂ |
| AcTE14011 | | |
| TF2: guanyl- | 33 | guanyl-Arg-Arg-Nal-Cys-Tyr-Cit-Lys-DGlu-Pro-Tyr-Arg-Cit-Cys-Arg- |
| TE14011 | | NH₂ |
| TF3: | 34 | TMguanyl-Arg-Arg-Nal-Cys-Tyr-Cit-Lys-DGlu-Pro-Tyr-Arg-Cit-Cys- |
| TMguanyl- | | Arg-NH₂ |
| TE14011 | | |
| TF4: | 35 | TMguanyl-Arg-Nal-Cys-Tyr-Cit-Lys-DGlu-Pro-Tyr-Arg-Cit-Cys-Arg- |
| TMguanyl- | | NH₂ |
| TE14011 (2- | | |
| 14) | | |
| TF5: 4F- | 36 | 4F-benzoyl-Arg-Arg-Nal-Cys-Tyr-Cit-Lys-DGlu-Pro-Tyr-Arg-Cit-Cys- |
| benzoyl- | | Arg-NH₂ |
| TE14011 | | |
| TF6: 2F- | 37 | 2F-benzoyl-Arg-Arg-Nal-Cys-Tyr-Cit-Lys-DGlu-Pro-Tyr-Arg-Cit-Cys- |
| benzoyl- | | Arg-NH₂ |
| TE14011 | | |
| TF7: APA- | 38 | APA-Arg-Nal-Cys-Tyr-Cit-Lys-DGlu-Pro-Tyr-Arg-Cit-Cys-Arg-NH₂ |
| TE14011 (2- | | |
| 14) | | |
| TF8: | 39 | desamino-R-Arg-Nal-Cys-Tyr-Cit-Lys-DGlu-Pro-Tyr-Arg-Cit-Cys-Arg- |
| desamino-R- | | NH₂ |
| TE14011 (2- | | |
| 14) | | |
| TF9: guanyl- | 40 | Guany1-Arg-Nal-Cys-Tyr-Cit-Lys-DGlu-Pro-Tyr-Arg-Cit-Cys-Arg-NH₂ |
| TE14011 (2- | | |
| 14) | | |
| TF10: | 41 | succinyl-Arg-Nal-Cys-Tyr-Cit-Lys-DGlu-Pro-Tyr-Arg-Cit-Cys-Arg-NH₂ |
| succinyl- | | |
| TE14011 (2- | | |
| 14) | | |
| TF11: | 42 | glutaryl-Arg-Nal-Cys-Tyr-Cit-Lys-DGlu-Pro-Tyr-Arg-Cit-Cys-Arg-NH₂ |
| glutaryl- | | |
| TE14011 (2- | | |
| 14) | | |
| TF12: | 43 | deaminoTMG-APA-Arg-Nal-Cys-Tyr-Cit-Lys-DGlu-Pro-Tyr-Arg-Cit- |
| deaminoTM | | Cys-Arg-NH₂ |
| G-APA- | | |
| TE14011 (2- | | |
| 14) | | |
| TF15: H- | 44 | R-CH2-Arg-Nal-Cys-Tyr-Cit-Lys-DGlu-Pro-Tyr-Arg-Cit-Cys-Arg-NH₂ |
| Arg-CH2NH- | | |
| RTE14011 | | |
| (2-14) | | |
| TF17: | 45 | H-Arg-Nal-Cys-Tyr-Cit-Lys-DGlu-Pro-Tyr-Arg-Cit-Cys-Arg-NH₂ |
| TE14011 (2- | | |
| 14) | | |
| TF18: | 46 | TMguanyl-Arg-Arg-Nal-Cys-Tyr-Cit-Lys-DCit-Pro-Tyr-Arg-Cit-Cys- |
| TMguanyl- | | Arg-NH₂ |
| TC14012 | | |
| TF19: ACA- | 47 | ACA-Arg-Arg-Nal-Cys-Tyr-Cit-Lys-DCit-Pro-Tyr-Arg-Cit-Cys-Arg- |
| TC14012 | | NH₂ |
| TF20: ACA- | 48 | ACA-Arg-Arg-Nal-Cys-Tyr-Arg-Lys-DLys-Pro-Tyr-Arg-Cit-Cys-Arg- |
| T140 | | OH |
| TZ14011 | 49 | H-Arg-Arg-Nal-Cys-Tyr-Cit-Arg-DLys-Pro-Tyr-Arg-Cit-Cys-Arg-NH₂ |
| AcTZ14011 | 50 | Ac-Arg-Arg-Nal-Cys-Tyr-Cit-Arg-DLys-Pro-Tyr-Arg-Cit-Cys-Arg-NH₂ |
| AcTN14003 | 51 | Ac-Arg-Arg-Nal-Cys-Tyr-Cit-Lys-DLys-Pro-Tyr-Arg-Cit-Cys-Arg-NH₂ |
| AcTN14005 | 52 | Ac-Arg-Arg-Nal-Cys-Tyr-Arg-Lys-DCit-Pro-Tyr-Arg-Cit-Cys-Arg-NH₂ |
| 4F-benzoyl- | 53 | 4F-benzoyl-Arg-Arg-Nal-Cys-Tyr-Cit-Lys-DGlu-Pro-Tyr-Arg-Cit-Cys- |
| TN14011-Me | | Arg-NHMe |
| 4F-benzoyl- | 54 | 4F-benzoyl-Arg-Arg-Nal-Cys-Tyr-Cit-Lys-DGlu-Pro-Tyr-Arg-Cit-Cys- |
| TN14011-Et | | Arg-NHEt |
| 4F-benzoyl- | 55 | 4F-benzoyl-Arg-Arg-Nal-Cys-Tyr-Cit-Lys-DGlu-Pro-Tyr-Arg-Cit-Cys- |
| TN14011-iPr | | Arg-NHiPr |
| 4F-benzoyl- | 56 | 4F-benzoyl-Arg-Arg-Nal-Cys-Tyr-Cit-Lys-DGlu-Pro-Tyr-Arg-Cit-Cys- |
| TN14011- | | Arg-tyramine |
| tyramine | | |
| TA14001 | 57 | H-Ala-Arg-Nal-Cys-Tyr-Arg-Lys-DLys-Pro-Tyr-Arg-Cit-Cys-Arg-OH |
| TA14005 | 58 | H-Arg-Arg-Nal-Cys-Tyr-Ala-Lys-DLys-Pro-Tyr-Arg-Cit-Cys-Arg-OH |
| TA14006 | 59 | H-Arg-Arg-Nal-Cys-Tyr-Arg-Ala-DLys-Pro-Tyr-Arg-Cit-Cys-Arg-OH |
| TA14007 | 60 | H-Arg-Arg-Nal-Cys-Tyr-Arg-Lys-DAla-Pro-Tyr-Arg-Cit-Cys-Arg-OH |
| TA14008 | 61 | H-Arg-Arg-Nal-Cys-Tyr-Arg-Lys-DLys-Ala-Tyr-Arg-Cit-Cys-Arg-OH |
| TA14009 | 62 | H-Arg-Arg-Nal-Cys-Tyr-Arg-Lys-DLys-Pro-Ala-Arg-Cit-Cys-Arg-OH |
| TA14010 | 63 | H-Arg-Arg-Nal-Cys-Tyr-Arg-Lys-DLys-Pro-Tyr-Ala-Cit-Cys-Arg-OH |
| TC14001 | 64 | H-Cit-Arg-Nal-Cys-Tyr-Arg-Lys-DLys-Pro-Tyr-Arg-Cit-Cys-Arg-OH |
| TC14003 | 65 | H-Arg-Arg-Nal-Cys-Tyr-Cit-Lys-DLys-Pro-Tyr-Arg-Cit-Cys-Arg-OH |
| TN14003 | 66 | H-Arg-Arg-Nal-Cys-Tyr-Cit-Lys-DLys-Pro-Tyr-Arg-Cit-Cys-Arg-NH₂ |
| TC14004 | 67 | H-Arg-Arg-Nal-Cys-Tyr-Arg-Cit-DLys-Pro-Tyr-Arg-Cit-Cys-Arg-OH |
| TC14012 | 68 | H-Arg-Arg-Nal-Cys-Tyr-Cit-Lys-DCit-Pro-Tyr-Arg-Cit-Cys-Arg-NH₂ |
| T-140 | 69 | H-Arg-Arg-Nal-Cys-Tyr-Arg-Lys-DLys-Pro-Tyr-Arg-Cit-Cys-Arg-OH |
| TC14011 | 70 | H-Arg-Arg-Nal-Cys-Tyr-Cit-Lys-DCit-Pro-Tyr-Arg-Cit-Cys-Arg-OH |
| TC14005 | 71 | H-Arg-Arg-Nal-Cys-Tyr-Arg-Lys-DCit-Pro-Tyr-Arg-Cit-Cys-Arg-OH |
| TC14018 | 72 | H-Cit-Arg-Nal-Cys-Tyr-Arg-Lys-DCit-Pro-Tyr-Arg-Cit-Cys-Arg-NH₂ |

According to a specific embodiment, in each one of SEQ ID NOS: 1-72, two cysteine residues are coupled in a disulfide bond.

In another embodiment, the peptide used in the compositions and methods of the invention consists essentially of an amino acid sequence as set forth in SEQ ID NO:1. In another embodiment, the peptide used in the compositions and methods of the invention comprises an amino acid sequence as set forth in SEQ ID NO:1. In another embodiment, the peptide is at least 60%, at least 70% or at least 80% homologous to SEQ ID NO:1. In another embodiment, the peptide is at least 90% homologous to SEQ ID NO:1. In another embodiment, the peptide is at least about 95% homologous to SEQ ID NO:1. Each possibility represents a separate embodiment of the present invention.

In various other embodiments, the peptide is selected from SEQ ID NOS: 1-72, wherein each possibility represents a separate embodiment of the present invention.

According to a specific embodiment, the peptide has an amino acid sequence as set forth in SEQ ID NO: 1 under the name Motixafortide.

As mentioned, the VLA-4 inhibitory (VLA4i) agent is Natalizumab [abbreviated herein as (N)].

According so some embodiment, the Natalizumab is sold under the brand name Tysabri®.

According so some embodiment, the Natalizumab is sold under the brand name Antegren®.

The peptide and VLA4i (“agents”) described hereinabove can be administered to the subject per se, or in pharmaceutical compositions being mixed with suitable carriers or excipients. Each of the agents can be formulated in a separate formulation.

As used herein a “pharmaceutical composition” refers to a preparation of one or more of active ingredients such as described herein with other chemical components such as physiologically suitable carriers and excipients. The purpose of a pharmaceutical composition is to facilitate administration of a compound to an organism.

Herein the term “active ingredient” refers to the agents accountable for the biological effect, e.g., SEQ ID NO: 1 or VLA4i.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, intradermal, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

According to a specific embodiment, the peptide of the invention or the pharmaceutical composition comprising same is administered subcutaneously (SC).

According to a specific embodiment, the VLA4i of the invention or the pharmaceutical composition comprising same is administered intravenously (IV).

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

Pharmaceutical compositions suitable for use in context of some embodiments of the invention include compositions wherein the active ingredients are contained in an amount effective to achieve the intended purpose. More specifically, according to specific embodiments, a therapeutically effective amount means on the one hand a mobilizing effective amount and on the other hand an amount of active ingredients effective to prevent, alleviate or ameliorate symptoms of a disorder, e.g., SCD.

According to specific embodiments the peptide of the invention or the pharmaceutical composition comprising same is administered in a dose ranging between 0.1 to 10 mg/kg of body weight, between 0.1 to 2 mg/kg of body weight, between 0.1 to 1 mg/kg of body weight, between 0.3 to 10 mg/kg of body weight, between 0.3 to 2.

According to a specific embodiment, BL-8040 is administered at a dose of 1-2 mg/kg body weight.

According to a specific embodiment, the, BL-8040 is administered at a dose of 1.25-1.5 mg/kg body weight.

According to a specific embodiment, the BL-8040 is administered at a dose of 1.25 mg/kg body weight.

According to a specific embodiment, the BL-8040 is administered subcutaneously (SC).

According to a specific embodiment, the Natalizumab is administered prior to said peptide.

According to a specific embodiment, the peptide is administered at a time point not exceeding 24 hours following administration of said Natalizumab.

According to a specific embodiment, the peptide is administered about 24 hours following administration of said Natalizumab.

According to a specific embodiment, the method comprises performing apheresis to retrieve said CD34+ cells from peripheral blood of the subject following administering the agents.

About 4 hours and up to about 12 hours, following administration of BL8040, the patient undergoes apheresis.

Methods of collecting peripheral blood are well known in the art and include, but not limited to drawing of up to 500 ml whole blood from the subject and collection in a container containing an anti-coagulant (e.g. heparin or citrate); and apheresis.

As used herein, the term “apheresis” refers to a procedure in which the peripheral blood of an individual is passed through an apparatus, yielding a predominant constituent (e.g. HSCs), and returning the other constituents to the subject's circulation. Apheresis is in general a three-step process comprising: (1) withdrawing blood from the subject, (2) separating the blood components (e.g. based on density), and (3) returning certain component(s) of the blood to the subject by transfusion. The blood is normally separated into three fractions: red blood cells (about 45% of total blood), “buffy coat’ (less than 1% of total blood) and plasma (about 55% of total blood). Various types of apheresis procedures can be used depending on the component of blood that is being removed.

As mentioned hereinabove, the collected fraction is subject to various research or diagnostic protocols such as transcription and immune-phenotyping.

The collected fraction can also be subject to treatments that can impart erythrocytes maturing from the red blood cells with a healthy phenotype.

Thus, according to an aspect of the invention there is provided a method of treating sickle cells disease (SCD), the method comprising:
(a) mobilizing CD34+ cells in a subject suffering from sickle cell disease (SCD) according to any one of claims 1-16;
(b) performing apheresis to retrieve said CD34+ cells from peripheral blood of the subject; and
(c) treating the CD34+ cells to obtain red blood cells with a healthy phenotype.

Thus, the cells used are autologous to the subject.

Following is a non-limiting summary of genetic approaches used to manipulate the collected cells (following apheresis).

The cells may be subject to genetic manipulation or first cultured.

For example, ex vivo culture with cytokine stimulation is typically required for lentiviral transduction in CD34+ HSCs. Typically, one-day pre-stimulation with serum-free culture media including cytokines (stem cell factor (SCF), FMS-like tyrosine kinase 3 ligand (FLT3L), and thrombopoietin (TPO), 100 ng/mL each) followed by one-day transduction with an LV allows for robust engraftment and efficient EGFP gene marking in CD34+ cells in xenograft mice. A lower concentration of SCF enhances the engraftment of CD34+ cells, and serum albumin can be replaced with polyvinyl alcohol. Long-term culture on fibronectin-coated plates allows for the engraftment of CD34+ cells. High-density culture with adjuvants such as dimethyl-prostaglandin E2 (PGE2) and amphiphilic drug-delivery poloxamers can improve transduction efficiency ~10-fold in human CD34+ cells in vitro. Overall, cytokine stimulation is required for lentiviral transduction in ex vivo CD34+ cell culture. However, minimal stimulation and short-term culture can also be used to maintain the balance between efficient gene addition and robust engraftment of CD34+ cells.

Therapeutic genes, such as wild-type β-globin, β^{T87Q}-globin (containing an anti-sickling mutation), or γ-globin are usually inserted into a self-inactivating (SIN) LV. Additionally, a β-globin promoter and the locus control region (LCR) are inserted to control transgene expression. As the 2nd intron of the β-globin gene is important to its expression, traditional vectors carry β-globin cassettes in the opposite direction of the vector genome to prevent transcriptional excision. Since this reversed construct reduces vector titers and transduction efficiency in CD34+ cells, a forward-oriented β-globin vector was recently developed. It addressed these issues and produced robust β-globin expression in erythroid cells.

Following are some clinical trials for SCD which are embodiments of the invention (Table 2 of J Clin Invest. 2021 Apr. 15; 131 (8): e146394).

TABLE 2
Autologous genetic therapies for SCD and β-thalassemia in clinical trials

| | | | | Clinical | | | |
|---|---|---|---|---|---|---|---|
| | | Lead | | trial | No. of | | |
| Strategy | Modality | Group | Status | number | participants | Results | References |
| B-Like | B^{A-T87Q} | Bluebird | Phase III, | NCT02140554, | 40 | 25 SCD (Group C) | 55, 210 |
| goblin | LV | bio | marketing | NCT02906202 | SCD, | participants | |
| gene | | | approval | | 23 β- | followed for 3-25 | |
| replacement | | | (Europe) | | thalassemia | months. Most had | |
| | | | | | | near-pancellular | |
| | | | | | | expression of | |
| | | | | | | HbA^{T87Q} ≥ 6 months | |
| | | | | | | after therapy with | |
| | | | | | | 99.5% mean | |
| | | | | | | reduction in the | |
| | | | | | | annualized | |
| | | | | | | VOC + ACS rate | |
| | | | | | | overall; 89% of | |
| | | | | | | evaluable | |
| | | | | | | participants with β- | |
| | | | | | | thalassemia are | |
| | | | | | | transfusion | |
| | | | | | | independent at a | |
| | | | | | | median of 19 | |
| | | | | | | months follow-up. | |
| | Modified | Cincinnati | Phase I/II | NCT02186418 | 3 | 3 SCD participants, | 154 |
| | y⁶¹⁶⁰- | Children's | | | | follow-up 6-30 | |
| | globin | Hospital | | | | months, with | |
| | LV | Medical | | | | clinical | |
| | | Center | | | | improvement | |
| | | | | | | (decreased VOEs) | |
| | Modified | CSL | Phase I | NCT04091737 | 3 | No results posted | |
| | y⁶¹⁵⁰- | Behring | | | | | |
| | globin | | | | | | |
| | LV | | | | | | |
| | LV | Assistance | Phase I/II | NCT03964792 | 10 | No results posted | |
| | expressing | Publique- | | | | | |
| | the | Hôpitaux | | | | | |
| | βAS3- | de Paris | | | | | |
| | globin | | | | | | |
| | gene | | | | | | |
| | βAS3- | University of | Phase I/II | NCT02247843 | 6 | No results posted | |
| | FB LV | California, | | | | | |
| | | Los | | | | | |
| | | Angeles | | | | | |
| HbF | BCL11A | Dana- | Phase I/II | NCT03282656 | 6 | 6 SCD participants | 33 |
| induction | shRNA | Farber | | | | infused, follow-up | |
| | LV | Cancer | | | | 7-29 months. No | |
| | | Institute | | | | patient has had a | |
| | | | | | | VOC, ACS, or | |
| | | | | | | stroke since the | |
| | | | | | | gene therapy | |
| | | | | | | infusion. | |
| | Cas9 | CRISPR | Phase I/II | NCT03745287 | 2 SCD, | 2 SCD participants | 32, 83 |
| | NHEJ; | Therapeutics/ | | | 5 β- | with follow-up of 3 | |
| | disruption of | Vertex | | | thalassemia | and 12 months | |
| | erythroid | Pharmaceuticals | | | | have had no SCD- | |
| | enhancer in | | | | | related VOEs since | |
| | BCL11A | | | | | infusion of gene- | |
| | gene | | | | | modified cells; 5 β- | |
| | | | | | | thalassemia | |
| | | | | | | participants with | |
| | | | | | | follow-up of 3-15 | |
| | | | | | | months are | |
| | | | | | | transfusion | |
| | | | | | | independent. | |
| | Cas9 | Novartis/ | Phase I/II | NCT04443907 | Not | No results posted | |
| | NHEJ | Intellia | | | Reported | | |
| | ZFN | Sangamo/ | Phase I/II | NCT03432364 | 2 | 2 participants with | 211 |
| | NHEJ; | Bioverativ/ | | | | β-thalassemia | |
| | disruption of | Sanofi | | | | infused, follow-up | |
| | erythroid | | | | | 3-6 months. Both | |
| | enhancer in | | | | | Require | |
| | BCL11A | | | | | intermittent | |
| | gene | | | | | transfusions, | |
| | | | | | | though | |
| | | | | | | requirements are | |
| | | | | | | decreased. | |

The manipulated cells can then be transfused back to the subject.

Each of which is contemplated herein. It is expected that during the life of a patent maturing from this application many relevant manipulations of CD34+ cells will be developed and the scope of the term “treating the CD34+” is intended to include all such new technologies a priori.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

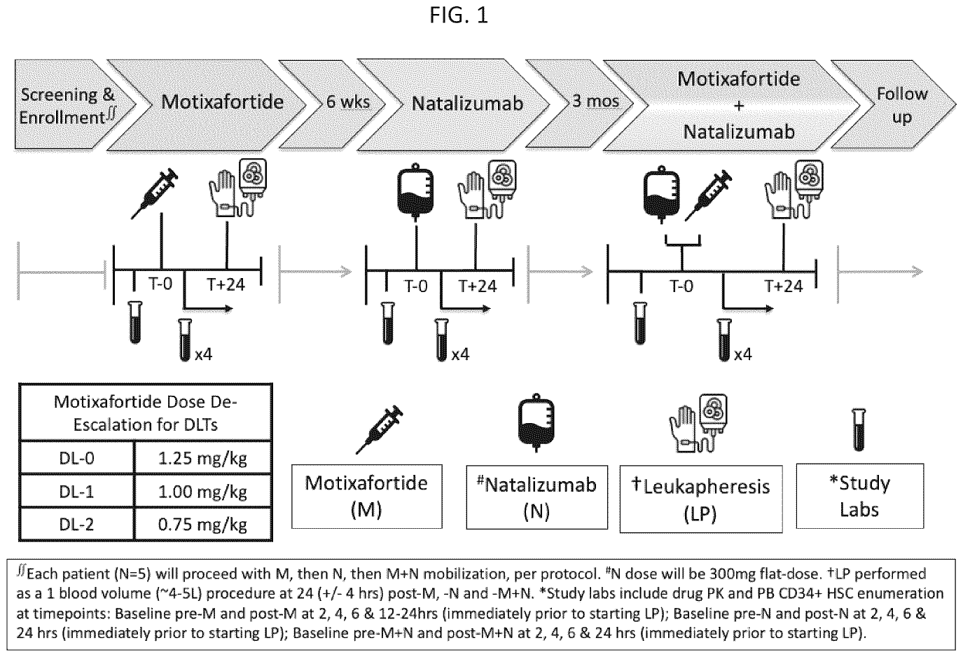

An outline of the treatment is shown in FIG. 1.

### Design

Single-arm, single center, open label pilot study.

### Study Population

N is 5 patients age 18-40 years with the diagnosis of SCD) hemoglobin SS or Sβ°) receiving automated RBC exchanges via apheresis-capable venus access.

### Key Eligibility Criteria

Able and willing to provide consent and meeting study population criteria above with ECOG ≤1 and adequate marrow/organ function.

### Primary Objective/Endpoint

Safety and tolerability of (M) and (N)+(M) in SCD patients defined by pre-specified dose limiting toxicities.

### Secondary Objective/Endpoints

Number of CD34+ cell mobilized via leukophoresis (CD34+ cells/kg/L) by total volume (tv) processed (1 blood volume (about 4-5 L procedure)).

Number of CD34+ cell mobilized via leukophoresis (CD34+ cells/kg/L) by adjusted volume (aV) processed (1 blood volume (about 4-5 L procedure)).

Kinetics of CD34+ HSPC PB mobilization (cells per microliter in PB).

Incidence of adverse events.

### Exploratory Objectives/Endpoints

Immunophenotypic and transcriptional profiling of PB CD34+ HSCs mobilized on study.

Gene editing efficiency and HSC fitness profiling of PB CD34+ HSCs mobilized on study.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of mobilizing CD34+ cells in a subject suffering from sickle cell disease (SCD), the method comprising administering to the subject a therapeutically effective amount of a CXCR4 inhibitory peptide as set forth in SEQ ID NO: 1 and a therapeutically effective amount of Natalizumab, wherein said Natalizumab and said CXCR4 inhibitory peptide are administered sequentially.

2. (canceled)

3. The method of claim 1, wherein said Natalizumab is administered intravenously (i.v.).

4. The method of claim 1, wherein said peptide is formulated as Motixafortide.

5. The method of claim 1, wherein said peptide is administered subcutaneously (S.C.).

6. The method of claim 5, wherein said peptide is administered at a dose of 0.1 to 10 mg/kg of body weight.

7. The method of claim 5, wherein said peptide is administered at a dose of 1-2 mg/kg of body weight.

8. The method of claim 5, wherein said peptide is administered at a dose of 1.25 mg/kg of body weight.

9. The method of claim 1, wherein said Natalizumab is administered intravenously (i.v.).

10. The method of claim 9, wherein said Natalizumab is administered at a dose of 100-1000 mg.

11. The method of claim 9, wherein said Natalizumab is administered at a dose of 300 mg.

12. The method of claim 1, wherein said Natalizumab is administered prior to said peptide.

13. The method of claim 1, wherein said peptide is administered at a time point not exceeding 24 hours following administration of said Natalizumab.

14. The method of claim 1, wherein said peptide is administered 24 hours following administration of said Natalizumab.

15. The method of claim 1, comprising performing apheresis to retrieve said CD34+ cells from peripheral blood of the subject following administering said agents.

16. A method of treating sickle cells disease (SCD), the method comprising:
(a) mobilizing CD34+ cells in a subject suffering from sickle cell disease (SCD) according to claim 1;
(b) performing apheresis to retrieve said CD34+ cells from peripheral blood of the subject; and
(c) treating the CD34+ cells to obtain red blood cells with a healthy phenotype.

17. The method of claim 16, wherein said treating is by genome editing.

18. The method of claim 16, wherein said healthy phenotype comprises expression of fetal hemoglobin (HbF; hemoglobin F).
